(19) United States
(12) Patent Application Publication
(10) Pub. No.: US 2026/0364015 A1
(43) Pub. Date: Nov. 19, 2026

(54) MIXED RELEASE PROFILE POLYMER MICROSPHERE FORMULATIONS COMPRISING OCTREOTIDE AND METHODS FOR MAKING AND USING THE SAME

(71) Applicant: OAKWOOD LABORATORIES, LLC, Oakwood Village, OH (US)

(72) Inventors: Audrey VILLAROSA, Solon, OH (US); Bradley MINROVIC, Willoughby, OH (US); Tracy RICHEY, Stow, OH (US); Regan SELLERS, Lakewood, OH (US); Matthew CORNELL, Mayfield Heights, OH (US)

(21) Appl. No.: 19/124,464

(22) PCT Filed: Oct. 31, 2023
(86) PCT No.: PCT/US2023/078271

§ 371 Date: Apr. 25, 2025

Related U.S. Application Data
(60) Provisional application No. 63/381,696, filed on Oct. 31, 2022.

(51) Int. Cl. A61K 9/16; A61K 9/00; A61K 38/08

(52) U.S. Cl. CPC A61K 9/1647; A61K 9/1682; A61K 38/08; A61K 9/0019

(57) ABSTRACT

Mixed release profiled polymer microsphere formulations comprising octreotide are provided. In one aspect, the microsphere formulations are characterized in that octreotide is released in vivo in humans over a period of about 60 days. Methods for making and using the formulations are also provided.

### CROSS-REFERENCE TO RELATED APPLICATIONS

This application claims priority from U.S. Provisional Patent Application No. 63/381,696, filed on Oct. 31, 2022, which is incorporated by reference herein in its entirety.

### BACKGROUND

Octreotide acetate (chemical formula C₅₃H₇₆N₁₀O₁₄S₂; CAS Number 79517-01-4), characterized by the general structure:

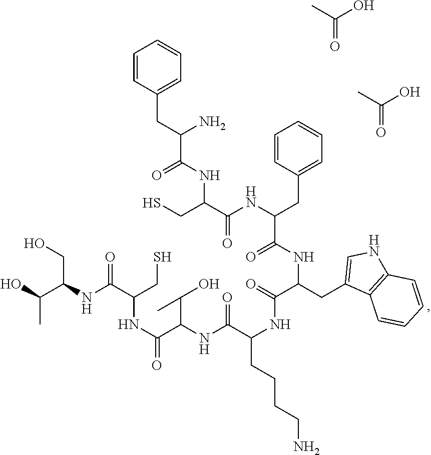

is used for the treatment of growth hormone-producing tumors (acromegaly and gigantism) when surgery is contraindicated, pituitary tumors t that secrete thyroid-stimulating hormone (thyrotropinoma), diarrhea and flushing episodes associated with carcinoid syndrome, and diarrhea in people with vasoactive intestinal peptide-secreting tumors.

Octreotide acetate is currently available under the brand name Sandostatin® as an immediate-release, subcutaneous injection, and under the brand name Sandostatin LAR® as a 28-day sustained-release, intramuscular injection. Sandostatin LAR® involves a complex formulation made by a complex manufacturing process. See U.S. Pat. Nos. 5,538,739 and 5,639,480.

A need exists for formulations comprising octreotide, including octreotide acetate formulations, that may be administered by a long-acting (e.g., about 60 days), sustained-release injection. In particular, a need exists for mixed release profile polymer microsphere formulations comprising octreotide, wherein a first formulation of polymer microspheres is characterized by a release of octreotide above a therapeutic level over a first period of time, and wherein a second formulation of polymer microspheres is characterized by a release of octreotide above a therapeutic level over a second period of time that is delayed relative to the first period of time.

### SUMMARY

Mixed release profile microsphere formulations comprising octreotide are provided. In one aspect, the mixed release profile microsphere formulations comprise: (i) first polymer microspheres that are characterized by a release of octreotide above a therapeutic level between about 5 days and about 40 days; and (ii) second polymer microspheres that are characterized by a release of octreotide above a therapeutic level between about 20 days and about 60 days. In one aspect, the first polymer microspheres are further characterized by a peak release of octreotide between about 15 days and about 25 days. In one aspect, the second polymer microspheres are further characterized by a peak release of octreotide between about 35 days and about 50 days.

In one aspect, the mixed release profile microsphere formulations may be made by a method, the method comprising: (A) mixing: (i) a first biodegradable polymer in a first polymer solvent system to form a first polymer solution; (ii) octreotide in a first drug solvent system to form a first octreotide solution; (iii) the first polymer solution with the first octreotide solution to form a first dispersed phase; (B) mixing: (i) a second biodegradable polymer in a second polymer solvent system to form a second polymer solution; (ii) octreotide in a second drug solvent system to form a second octreotide solution; (iii) the second polymer solution with the second octreotide solution to form a second dispersed phase; (C) mixing: (i) water; and (ii) a surfactant, to form a continuous phase; (D) combining the first dispersed phase with a first portion of the continuous phase in a first homogenizer to form a first microsphere formulation having a first release profile; (E) combining the second dispersed phase with a second portion of the continuous phase in a second homogenizer to form a second microsphere formulation having a second release profile; and (F) combining the first microsphere formulation with the second microsphere formulation to form the mixed release profile microsphere formulation. In one aspect, the first and second homogenizers are the same.

In another aspect, single polymer microsphere formulations are provided, the single polymer microsphere formulations comprising polymer microspheres, each polymer microsphere comprising: (i) octreotide; and (ii) a biodegradable polymer, wherein each polymer microsphere comprises a drug load of octreotide of between about 6% and about 14% by weight of the polymer microsphere, and wherein the polymer microspheres have a particle size of between about 55 μm and about 75 μm (D₅₀). In one aspect, the microsphere formulations are characterized in that they have an in vivo duration of release of about 60 days in humans.

In one aspect, the single polymer microsphere formulations may be made by a method, the method comprising: (A) mixing: (i) a biodegradable polymer in a polymer solvent system to form a polymer solution; (ii) octreotide in a drug solvent system to form an octreotide solution; and (iii) the polymer solution with the octreotide solution to form a dispersed phase; (B) mixing: (i) water; and (ii) a surfactant, to form a continuous phase; and (C) combining the dispersed phase with the continuous phase in a homogenizer.

In one aspect, a method for treating acromegaly or diarrhea is provided. The method may comprise administering by intramuscular injection to a patient in need thereof a mixed release profile or single polymer microsphere formulation made according to the methods described herein.

In another aspect, use is disclosed of a mixed release profile or single polymer microsphere formulation comprising polymer microspheres, each polymer microsphere comprising: (i) octreotide; and (ii) a biodegradable polymer, wherein each polymer microsphere comprises a drug load of octreotide of between about 6% and about 14% by weight of the polymer microsphere, and wherein the polymer microspheres have a particle size of between about 50 μm and about 90 μm (D₅₀), in the manufacture of a medicament for the treatment of acromegaly or diarrhea.

In another aspect, a mixed release profile or single polymer microsphere formulation comprising polymer microspheres, each polymer microsphere comprising: (i) octreotide; and (ii) a biodegradable polymer, wherein each polymer microsphere comprises a drug load of octreotide of between about 6% and about 14% by weight of the polymer microsphere, and wherein the polymer microspheres have a particle size of between about 50 μm and about 90 μm (D₅₀), is provided for use as a medicament for the treatment of acromegaly or diarrhea.

In another aspect, a kit is provided, the kit comprising mixed release profile or single polymer microspheres, each mixed release profile or single polymer microsphere comprising: (i) octreotide; and (ii) a biodegradable polymer, wherein each polymer microsphere comprises a drug load of octreotide of between about 6% and about 14% by weight of the polymer microsphere, and wherein the polymer microspheres have a particle size of between about 50 μm and about 90 μm (D₅₀).

### BRIEF DESCRIPTION OF THE FIGURES

FIG. 1 is a schematic depicting an example system and method for making an octreotide-encapsulated, single polymer microsphere formulation.

FIG. 2 is a schematic depicting an example system and method for making an octreotide-encapsulated, mixed release profile polymer microsphere formulation, where the mixed release profile polymer microsphere formulation is combined and vialed via a single filling needle.

FIG. 3 is a schematic depicting an example system and method for making an octreotide-encapsulated, mixed release profile polymer microsphere formulation, where the mixed release profile polymer microsphere formulation is vialed via two separate suspension filles (two filling needles).

FIG. 4 is a graph showing in vitro cumulative octreotide release over time from four different formulations of octreotide-encapsulating polymer microspheres.

FIG. 5 is a graph showing mixed release profile dose modeling of octreotide-encapsulating polymer microspheres.

FIG. 6 is a graph showing the results of an in vivo pharmacokinetics study of the four formulations referred to in FIG. 4 in dogs.

FIG. 7 is a graph showing the results of an in vivo pharmacokinetics study of three single polymer microsphere formulations referred to in FIG. 4 in dogs.

FIG. 8 is a graph showing the results of an in vivo pharmacokinetics study of a single polymer microsphere formulation vs. a mixed release profile polymer microsphere formulation referred to in FIG. 4 in dogs.

FIG. 9 is a graph showing an amount of octreotide acetate released in vitro over time from small and large scale batches of one month octreotide acetate-encapsulated polymer microspheres of a mixed release profile polymer microsphere formulation.

FIG. 10 is a graph showing an amount of octreotide acetate released in vitro over time from small and large scale batches of two month octreotide acetate-encapsulated polymer microspheres of a mixed release profile polymer microsphere formulation.

### DETAILED DESCRIPTION

Mixed release profile microsphere formulations comprising octreotide are provided. In one aspect, the mixed release profile microsphere formulations comprise: (i) first polymer microspheres that are characterized by a release of octreotide above a therapeutic level between about 5 days and about 40 days; and (ii) second polymer microspheres that are characterized by a release of octreotide above a therapeutic level between about 20 days and about 60 days. In one aspect, the first polymer microspheres are further characterized by a peak release of octreotide between about 15 days and about 25 days. In one aspect, the second polymer microspheres are further characterized by a peak release of octreotide between about 35 days and about 50 days.

In one aspect, the mixed release profile microsphere formulations may be made by a method, the method comprising: (A) mixing: (i) a first biodegradable polymer in a first polymer solvent system to form a first polymer solution; (ii) octreotide in a first drug solvent system to form a first octreotide solution; (iii) the first polymer solution with the first octreotide solution to form a first dispersed phase; (B) mixing: (i) a second biodegradable polymer in a second polymer solvent system to form a second polymer solution; (ii) octreotide in a second drug solvent system to form a second octreotide solution; (iii) the second polymer solution with the second octreotide solution to form a second dispersed phase; (C) mixing: (i) water; and (ii) a surfactant, to form a continuous phase; (D) combining the first dispersed phase with a first portion of the continuous phase in a first homogenizer to form a first microsphere formulation having a first release profile; (E) combining the second dispersed phase with a second portion of the continuous phase in a second homogenizer to form a second microsphere formulation having a second release profile; and (F) combining the first microsphere formulation with the second microsphere formulation to form the mixed release profile microsphere formulation. In one aspect, the first and second homogenizers are the same.

### Octreotide

The active pharmaceutical ingredient is octreotide. The octreotide may be in a free base, acid addition salt, or complex form. In one aspect, the octreotide is an acetate salt. In one aspect, the octreotide acetate is supplied by Bachem. In one aspect, the octreotide acetate is hydrophilic. In one aspect, the octreotide acetate is characterized by an aqueous solubility of >220 mg/mL.

### Biodegradable Polymers

In one aspect, the dispersed phase may include a biodegradable polymer, such as a poly(lactic-co-glycolic acid) (a PLGA) or a polylactic acid (a PLA), although it is contemplated that other suitable biodegradable polymers may be used.

In some aspects, the biodegradable polymer comprises a PLGA. In one aspect, the PLGA comprises a lactide: glycolide ratio of 75:25 or 85:15. In an aspect where the microsphere formulation is a mixed release profile formulation, a first PLGA may comprise a lactide: glycolide ratio of 75:25, and a second PLGA may comprise a lactide: glycolide ratio of 85:15. In one aspect, where the microsphere formulation is a mixed release profile formulation, the biodegradable polymer consists of: (i) a first PLGA that consists of a lactide: glycolide ratio of 75:25, and (ii) a second PLGA that consists of a lactide: glycolide ratio of 85:15. For the avoidance of doubt, the mixed release profile formulation does not include a blend of PLGAs; rather, the mixed release profile polymer microsphere formulation comprises: (i) first polymer microspheres, each first polymer microsphere comprising: octreotide or a pharmaceutically acceptable salt thereof; and a first PLGA having a lactide: glycolide ratio, wherein each first polymer microsphere comprises a drug load of octreotide and a first particle size, wherein the first polymer microspheres are characterized by a release of octreotide above a therapeutic level for a first period; and (ii) second polymer microspheres, each second polymer microsphere comprising: octreotide or a pharmaceutically acceptable salt thereof; and a second PLGA having a lactide: glycolide ratio, wherein each second polymer microsphere comprises a drug load of octreotide and a second particle size, wherein the second polymer microspheres are characterized by a release of octreotide above a therapeutic level for a second period.

In one aspect, the PLGA is acid-terminated. In one aspect, ester-terminated PLGAs are specifically excluded. In one aspect, the PLGA has an IV of from about 0.1 dL/g to about 0.4 dL/g, including from about 0.1 dL/g to about 0.3 dL/g, from about 0.2 dL/g to about 0.4 dL/g, about 0.19 dL/g, about 0.2 dL/g, about 0.21 dL/g, and about 0.27 dL/g, and any value or range between any two of those IV values.

In one aspect, the PLGA comprises Viatel™ DLG 7502 A, poly(D,L-lactide-co-glycolide), acid terminated, lactide: glycolide 75:25, manufactured by Ashland, having IV=0.2 (“7502 A”). In one aspect, the PLGA comprises Viatel™ DLG 8502 A, poly(D,L-lactide-co-glycolide), acid terminated, lactide: glycolide 85:15, manufactured by Ashland, having IV=0.19 (“8502 A”). In one aspect, the PLGA comprises Viatel™ DLG 8503 A, poly(D,L-lactide-co-glycolide), acid terminated, lactide: glycolide 85:15, manufactured by Ashland, having IV=0.27 (“8503 A”).

In some aspects, the biodegradable polymer is a PLA. In one aspect, the PLA is acid-terminated. In one aspect, the PLA has an IV of between about 0.16 dL/g and about 0.24 dL/g, including about 0.21 dL/g, and any value or range between any two of those IV values.

In one aspect, the PLA comprises a Resomer® R 202 H, poly(D,L-lactide), acid terminated, manufactured by Evonik, having IV=0.21 (“202 H”).

### Dispersed Phase

In one aspect, the dispersed phase comprises a polymer solvent system. In one aspect, the polymer solvent system comprises dichloromethane (DCM). In one aspect, the dispersed phase comprises a drug solvent system. In one aspect, the drug solvent system comprises methanol (MeOH) and/or acetic acid (AcOH). In one aspect, the polymer is dissolved in the polymer solvent system, the octreotide is dissolved in the drug solvent system, and the two solutions are mixed prior to contacting with the continuous phase. In one aspect, the ratio of AcOH to octreotide may be 0.1:1, 0.2:1, 0.3:1, 0.4:1, 0.5:1, 0.6:1, 0.7:1, 0.8:1, 0.9:1 or about 1:1. In one aspect, the dispersed phase may include an antioxidant. In one aspect, the antioxidant may comprise butylated hydroxytoluene. The methods described herein do not include precipitating the dispersed phase by contacting it with anhydrous diethyl ether and/or anhydrous n-heptane.

### Continuous Phase

The dispersed phase may be combined with an aqueous continuous phase that comprises water and, optionally, a buffer, a surfactant, or both. A continuous phase comprising an organic ion selected from the group consisting of trifluoromethyl-p-toluate, 2-naphthalene sulfonate, 2,3-naphthalene dicarboxylate, 2-naphthoate, and salicylsalicylate is specifically excluded.

In one aspect, the buffer may be added to the continuous phase to maintain a pH of the solution of about 7.0 to about 8.0. In one aspect, the buffer may be a phosphate buffer or a carbonate buffer. In one aspect, the buffer may be a phosphate or carbonate buffer solution and may be used to create and maintain a system pH level of about 7.4.

The surfactant component may be present in the continuous phase in an amount of about 0.35% to about 1.0% by weight in water. In one aspect, the surfactant component comprises polyvinyl alcohol (“PVA”) in a concentration of 0.35% by weight in water. In one aspect, the surfactant component comprises polyvinyl alcohol (“PVA”) in a concentration of 1% by weight in water.

In some aspects, the dispersed phase flow rate to the homogenizer may be from about 10 mL/min to about 30 mL/min, including about 20 mL/min and about 25 mL/min. In some aspects, the continuous phase flow rate to the homogenizer may be about 2 L/min. Thus, in one aspect, the continuous phase: dispersed phase ratio may be from about 66:1 to about 200:1, including about 100:1 and about 80:1. Larger scale batches may require higher flow rates. For example, in some aspects, the flow rates may be doubled.

The continuous phase may be provided at room temperature or above or below room temperature. In some aspects, the continuous phase may be provided at about 40 °C, about 37 °C, about 35 °C, about 30 °C, about 25 °C, about 20 °C, about 15 °C, about 10 °C, about 5 °C, about 0 °C, and any value or range between any two of those temperature values.

### Homogenizer

For brevity, and because the methods are equally applicable to either, the phrase “homogenizer” contemplates a system or apparatus that can homogenize the dispersed phase and the continuous phase, emulsify the dispersed phase and the continuous phase, or both, which systems and apparatuses are known in the art. For example, in one aspect, the homogenizer is an in-line Silverson Homogenizer (commercially available from Silverson Machines, Waterside UK) used, e.g., as described in U.S. Pat. No. 5,945,126, or a Levitronix® BPS-i100 integrated pump system used, e.g., as described in U.S. Pat. No. 11,167,256, each of which is incorporated by reference herein in its entirety. In one aspect, the homogenizer is a membrane emulsifier or a static mixer. In one aspect, the homogenizer runs at an impeller speed of about 1,000 to about 4,000 revolutions per minute (RPM), including about 2,000 RPM, about 2,250 RPM, about 2,500 RPM, and any value or range between any two of those RPM values.

### Drug Load

The drug load of each polymer microsphere in a drug to polymer ratio, expressed as a percentage, may be from about 6% wt/wt % to about 14% wt/wt %, including about 7 wt/wt %, about 8 wt/wt %, about 9 wt/wt %, about 10 wt/wt %, about 11 wt/wt %, about 12 wt/wt %, and about 13 wt/wt %, and any value or range between any two of those drug loads.

### Particle Size

In one aspect, the polymer microspheres may have a particle size of less than 90 μm (D₅₀), including between about 55 μm (D₅₀) and about 75 μm (D₅₀), between about 60 μm (D₅₀) and about 75 μm (D₅₀), about 60 μm (D₅₀), about 65 μm (D₅₀), about 70 μm (D₅₀), about 75 μm (D₅₀), and any value or range between any two of those particle sizes.

### Extended Release

In one aspect, the microsphere formulations are characterized in that they have an in vivo duration of release of about 60 days in humans, i.e., plasma levels of octreotide are maintained within the therapeutic range for a period of about 60 days after injection. In one aspect, the microsphere formulations comprise a mixed release profile formulation. In one aspect, the mixed release profile formulation comprises: (i) first polymer microspheres that are characterized by a release of octreotide above a therapeutic level between about 5 days and about 40 days; and (ii) second polymer microspheres that are characterized by a release of octreotide above a therapeutic level between about 20 days and about 60 days. In one aspect, the first polymer microspheres are further characterized by a peak release of octreotide between about 15 days and about 25 days. In one aspect, the second polymer microspheres are further characterized by a peak release of octreotide between about 35 days and about 50 days. In one aspect, the microsphere formulations are characterized in that they have an in vivo duration of release such that plasma levels of octreotide are below the therapeutic range prior to three months after injection.

### Therapeutic Benefits

In one aspect, a method for treating acromegaly or diarrhea is provided. The method may comprise administering by intramuscular or subcutaneous injection to a patient in need thereof a single polymer microsphere formulation or a mixed release profile polymer microsphere formulation made according to the methods described herein.

In another aspect, use is disclosed of a single polymer microsphere formulation or a mixed release profile polymer microsphere formulation comprising polymer microspheres, each polymer microsphere comprising: (i) octreotide; and (ii) a biodegradable polymer, wherein each polymer microsphere comprises a drug load of octreotide of about 6% to about 14% by weight of the polymer microsphere, and wherein the polymer microspheres have a particle size of between about 55 μm and about 75 μm (D₅₀), in the manufacture of a medicament for the treatment of acromegaly or diarrhea.

In another aspect, a single polymer microsphere formulation or a mixed release profile polymer microsphere formulation comprising polymer microspheres, each polymer microsphere comprising: (i) octreotide; and (ii) a biodegradable polymer, wherein each polymer microsphere comprises a drug load of octreotide of about 6% to about 14% by weight of the polymer microsphere, and wherein the polymer microspheres have a particle size of between about 55 μm and about 75 μm (D₅₀), is provided for use as a medicament for the treatment of acromegaly or diarrhea.

In another aspect, a kit is provided, the kit comprising a single polymer microsphere formulation or a mixed release profile polymer microsphere formulation comprising polymer microspheres, each polymer microsphere comprising: (i) octreotide; and (ii) a biodegradable polymer, wherein each polymer microsphere comprises a drug load of octreotide of about 6% to about 14% by weight of the polymer microsphere, and wherein the polymer microspheres have a particle size of between about 55 μm and about 75 μm (D₅₀).

### EXAMPLES

### Example 1—General Preparation of a Single Polymer Microsphere Formulation Comprising Octreotide Acetate

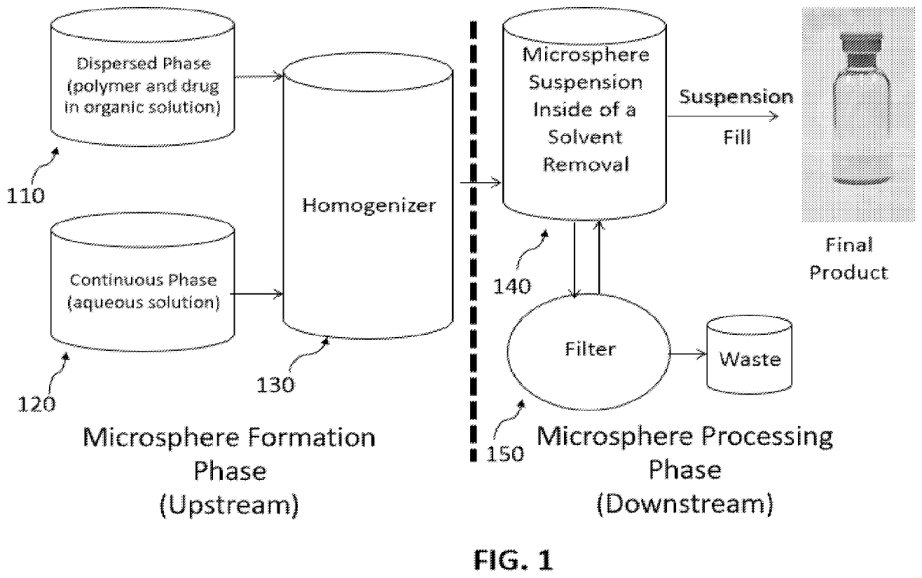

Microsphere Formation Phase. With reference to FIG. 1, a dispersed phase (“DP”) 110 is formed by dissolving a polymer matrix (such as a PLGA or PLA polymer) in a polymer solvent system (such as DCM), dissolving octreotide acetate in a drug solvent system (such as MeOH/AcOH), and mixing the two solutions. The DP 110 is filtered and pumped into a homogenizer 130 at a defined flow rate. A continuous phase (“CP”) 120 comprising water, surfactant, and, optionally, a buffer is also pumped into the homogenizer 130 at a defined flow rate. The speed of the homogenizer 130 is generally fixed to achieve a desired polymer microsphere size distribution. A representative continuous “upstream” microsphere formation phase is described in U.S. Pat. No. 5,945,126, which is incorporated by reference herein in its entirety.

Microsphere Processing Phase. The formed or forming microspheres exit the homogenizer 130 and enter a solvent removal vessel (“SRV”) 140. Water may be added to the SRV 140 during microsphere formation to minimize the solvent level in the aqueous medium. See, e.g., U.S. Pat. No. 9,017,715, which is incorporated by reference herein in its entirety. After the DP 110 has been exhausted, the CP 120 and water flow rates are stopped, and the washing steps are initiated. Solvent removal is achieved using water washing and a hollow fiber filter (commercially available as HFF from, e.g., Cytiva) 150. A microsphere is “essentially free” of organic solvent if the microsphere meets the standards set forth in the “ICH Harmonised Guideline, Impurities: Guideline for Residual Solvents Q3C (R8), Current Step 4 version dated 22 Apr. 2021,” which is incorporated herein by reference in its entirety. A representative “downstream” microsphere processing phase is described in U.S. Pat. No. 6,270,802, which is incorporated by reference herein in its entirety.

The washed microspheres are collected and freeze-dried in a lyophilizer (Virtis) to remove any moisture. The resulting microspheres are a free-flowing off-white bulk powder.

### Example 2—General Preparation of a Mixed Release Profile Polymer Microsphere Formulation Comprising Octreotide Acetate

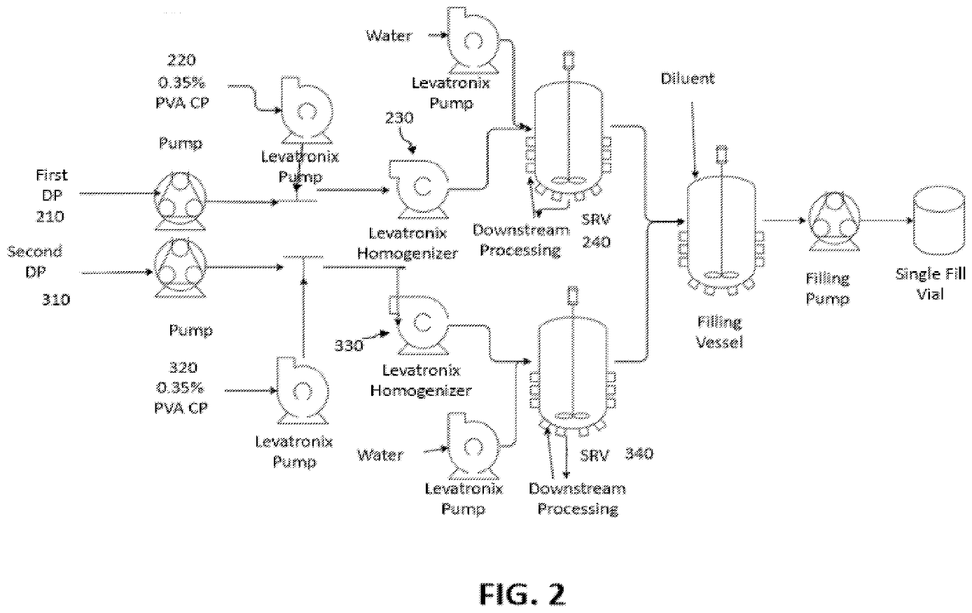

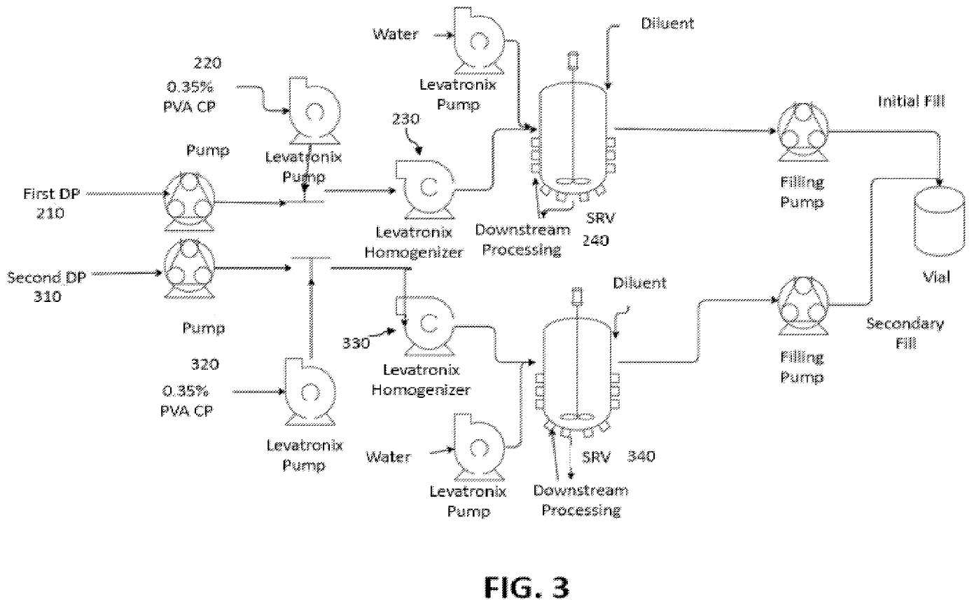

Microsphere Formation Phase. With reference to FIGS. 2 and 3, a first DP 210 is formed by dissolving a first polymer matrix (such as a PLGA or PLA polymer) in a first polymer solvent system (such as DCM), dissolving octreotide acetate in a first drug solvent system (such as MeOH/AcOH), and mixing the two solutions. A second DP 310 is formed by dissolving a second polymer matrix (such as a PLGA or PLA polymer) in a second polymer solvent system (such as DCM), dissolving octreotide acetate in a second drug solvent system (such as MeOH/AcOH), and mixing the two solutions. Each of the first DP 210 and the second DP 310 is filtered using a 0.2 μm sterilizing PTFE or PVDF membrane filter (such as EMFLON, commercially available from Pall or SartoriousAG), and each is pumped into a homogenizer 230/330 at a defined flow rate. A CP 220/320 comprising water, surfactant, and, optionally, a buffer is also pumped into the homogenizers 230 and 330 at a defined flow rate. The speed of the homogenizers 230 and 330 are generally fixed to achieve a desired polymer microsphere size distribution.

Microsphere Processing Phase. The formed or forming microspheres exit the homogenizers 230 and 330 and enter solvent removal vessels (“SRV”) 240 and 340, respectively. Water may be added to the SRVs 240 and 340 during microsphere formation to minimize the solvent level in the aqueous medium. After the DPs 210 and 310 have been exhausted, the CPs 220 and 320 and water flow rates are stopped, and washing steps are initiated. Solvent removal is achieved using water washing and a hollow fiber filter (commercially available as HFF from, e.g., Cytiva) (not shown).

### Example 3—Preparation of Octreotide Acetate-Encapsulated Polymer Microspheres

Groups 1, 2, and 4 (single polymer microsphere formulations) were prepared following the general procedure described in Example 1 and illustrated in FIG. 1, and Group 3 (a mixed release profile polymer microsphere formulation) was prepared following the general procedure described in Example 2 and illustrated in FIG. 2 and FIG. 3 (which are identical prior vialing). The specific processing parameters and microsphere physical characteristics are detailed in Table 1.

TABLE 1

| | | Group Number/Lot No. | | | | |
|---|---|---|---|---|---|---|
| | | Group 1/ | Group 2/ | Group 3/ | | Group 4/ |
| | | Lot 92 | Lot 93 | Lots 100&93 | | Lot 95 |
| | | Batch Purpose | | | | |
| | | | | Mixed Release Profile | | Single |
| | | Single Formulations | | Formulation | | Formulation |
| | | Approach | | | | |
| | | Lower IV | Higher IV | Month 1 | Month 2 | PLA |
| Symbol | | ◯ | Δ | □ | | ⋄ |
| (FIGS. 3-7) | | | | | | |
| Co-Monomer | | 85:15 | | 75:25 | 85:15 | 100:0 |
| Ratio | | | | | | |
| Polymer IV | | 0.19 | 0.27 | 0.20 | 0.27 | 0.21 |
| (dL/g) | | | | | | |
| Polymer | | Acid | | | | |
| Endcap | | | | | | |
| Mixing Speed | | 2000 | 2250 | 2500 | 2250 | 2000 |
| (RPM) | | | | | | |
| Target Drug | | 14 | | 10 | 14 | 14 |
| Load (%) | | | | | | |
| Drug Load | | 10.7 | 10.8 | 9.4 | | 10.5 |
| (%) | | | | | | |
| Encapsulation | | 76 | 77 | 78 | | 75 |
| Efficiency (%) | | | | | | |
| Particle | Dᵥ10 | 41 | 43 | 34 | 43 | 33 |
| Size | Dᵥ50 | 70 | 74 | 67 | 74 | 59 |
| (μm) | Dᵥ90 | 110 | 117 | 110 | 117 | 96 |

| | | | | | |
|---|---|---|---|---|---|
| Sample MW | 18.2 | 27.1 | 16.0 | 27.1 | 15.3 |
| (kDa) | | | | | |
| Polymer MW | 18.4 | 27.9 | 16.3 | 27.9 | 15.5 |
| (kDa) | | | | | |

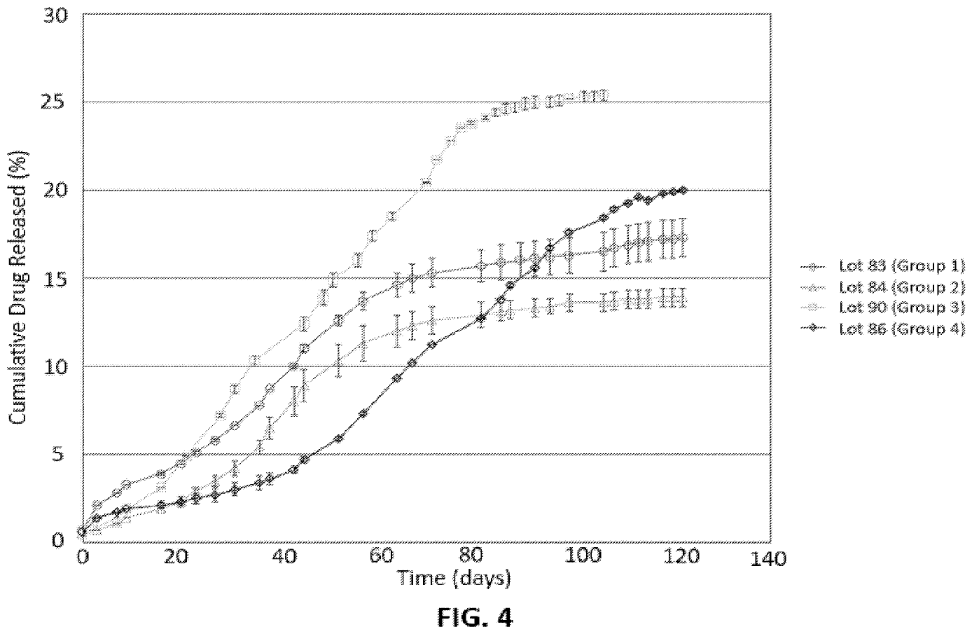

FIG. 4 is a graph showing an amount of octreotide acetate released in vitro over time from octreotide acetate-encapsulated polymer microsphere Groups 1-4.

### Example 4-Pharmacokinetics Study of Groups 1~4 in Dogs

The pharmacokinetic profile of octreotide acetate following an intramuscularly injected dose of time-released octreotide acetate formulations in dogs was studied. Two male and two female dogs per group were dosed as follows:
Octreotide per vial:
   Single polymer microsphere formulations: 110 mg octreotide/vial
   Mixed release profile polymer microsphere formulation: 220 mg octreotide/vial
Dose level:
   Single polymer microsphere formulations: 1 mg/kg @ 10 mg/mL octreotide concentration
   Mixed release profile polymer microsphere formulation: 2 mg/kg @ 10 mg/mL octreotide concentration
Dose volume:
   Single polymer microsphere formulations: 0.1 mL/kg
   Mixed release profile polymer microsphere formulation: 0.2 mL/kg
Blood samples:
   Pre-dose, 0.25 h, 0.5 h, 1 h, 2 h, 6 h, 24 h, 48 h, 72 h, 96 h, 168 h, every 7 days.

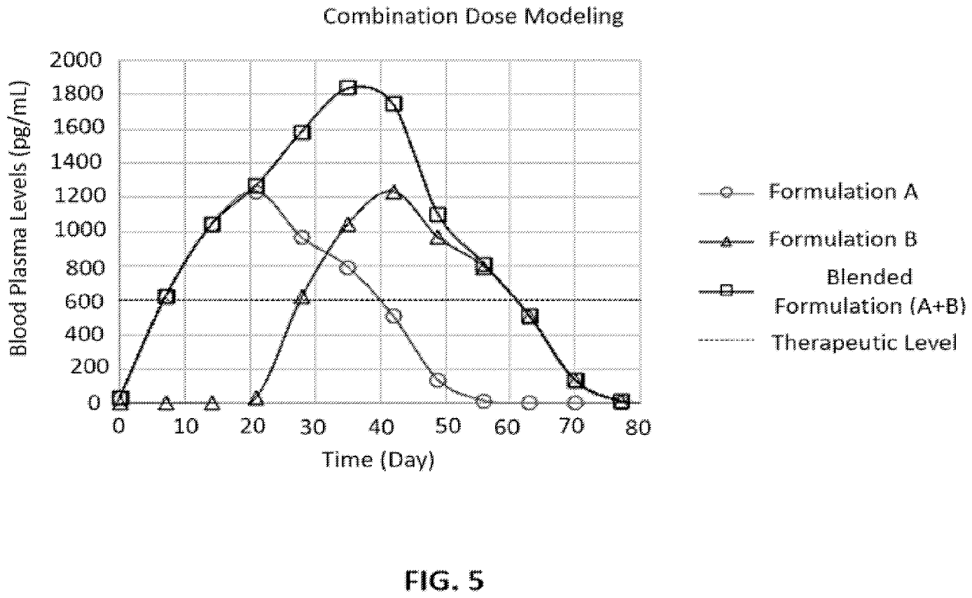

FIG. 5 is a graph showing mixed release profile dose modeling. Only group 3 is a mixed release profile formulation. Conceptually, the mixed release profile formulation comprises: (i) first polymer microspheres that are characterized by a release of octreotide acetate above a therapeutic level between about 5 days and about 40 days, with a peak release between about 15 days and about 25 days (Month 1, as shown in Table 1 and on FIG. 5); and (ii) second polymer microspheres that are characterized by a release of octreotide acetate above a therapeutic level between about 20 days and about 60 days, with a peak release between about 35 days and about 50 days (Month 2, as shown in Table 1 and on FIG. 5).

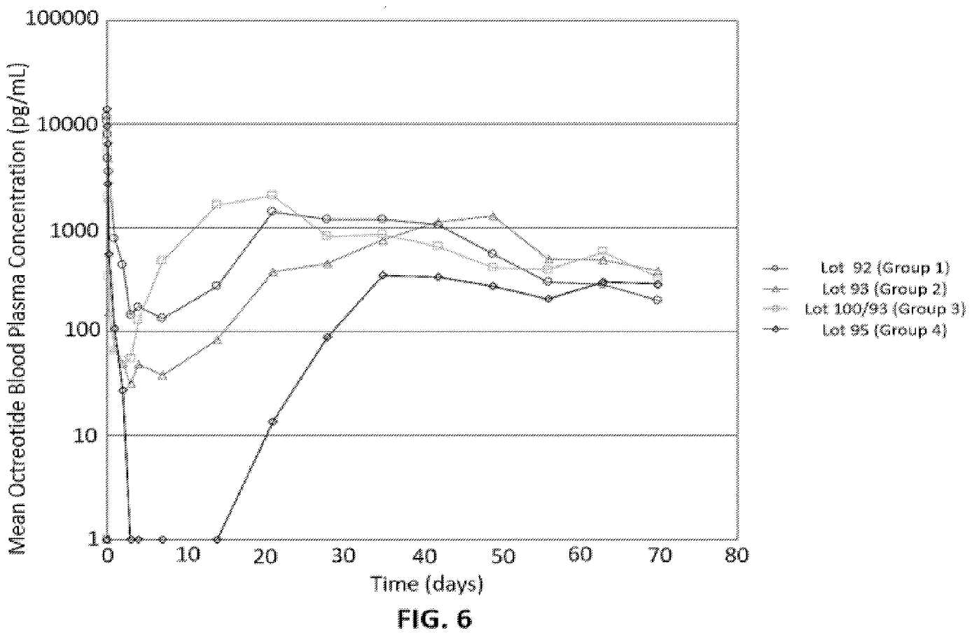

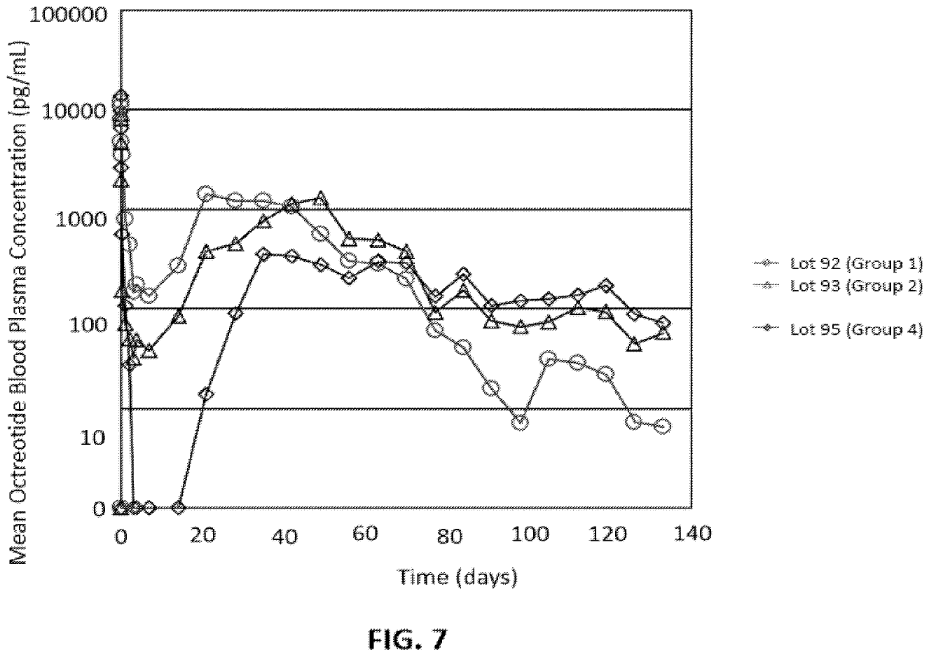

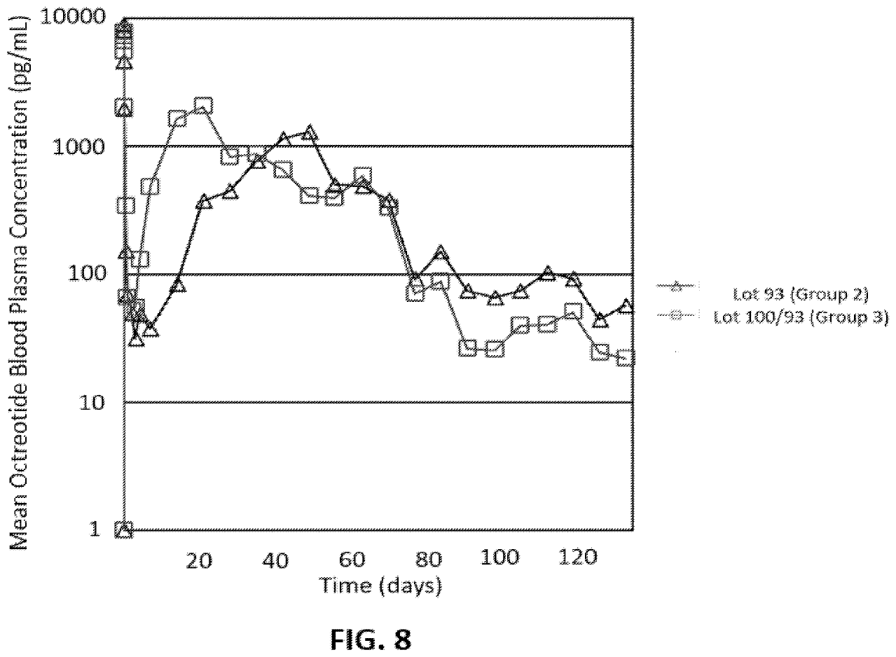

FIG. 6 is a log scale graph showing the measured mean blood concentration (ng/ml) of octreotide acetate as a function of time for groups 1-4 over the course of approximately 70 days. FIG. 7 shows the results of the three single polymer microsphere formulations, 1, 2, and 4, over 140 days. FIG. 8 shows the results of the Group 2 single polymer microsphere formulation vs. the Group 3 mixed release profile polymer microsphere formulation over 140 days.

### Example 5—Scale-Up of Octreotide Acetate-Encapsulated Polymer Microspheres

The one month formulation of the Group 3 blend was prepared following the general procedure described in Example 1 and illustrated in FIG. 1. A larger (250 g) batch was prepared in the same manner. The specific processing parameters and microsphere physical characteristics are detailed in Table 2.

TABLE 2

| | |
|---|---|
| Batch Purpose | Month One of Blend |

| | | |
|---|---|---|
| Batch No. | Lot 100 | Lot 153 |
| Batch Size | 20 g batch | 250 g batch |
| Symbol | ⋄ | ◯ |

| | |
|---|---|
| Co-Monomer | 75:25 |
| Ratio | |
| Polymer IV | 0.20 |
| (dL/g) | |
| Polymer | Acid |
| Endcap | |
| Mixing Speed | 2500 |
| RPM | |
| Target Drug | 10 |
| Load (%) | |

| Drug Load | | | 7.3 | 8.3 |
|---|---|---|---|---|
| (%) | | | | |
| Encapsulation | | | 73 | 83 |
| Efficiency (%) | | | | |
| | Particle | Dᵥ10 | 34 | 19 |
| | Size | Dᵥ50 | 67 | 64 |
| | (μm) | Dᵥ90 | 110 | 112 |

| | | |
|---|---|---|
| Sample MW | 16.0 | 19.8 |
| (kDa) | | |
| Polymer MW | 16.3 | 20.7 |
| (kDa) | | |

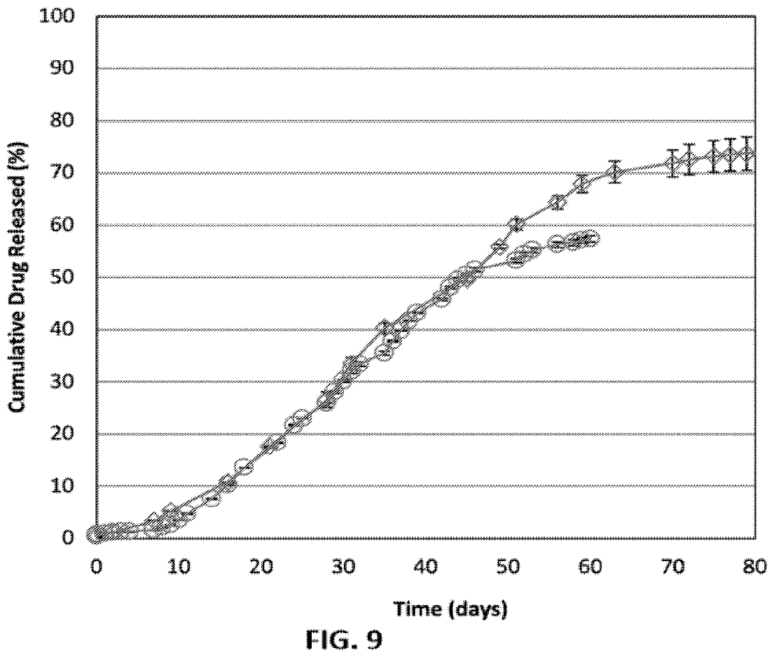

FIG. 9 is a graph showing an amount of octreotide acetate released in vitro over time from the smaller and larger scale batches of month one octreotide acetate-encapsulated polymer microspheres.

The two-month formulation of the Group 3 blend was prepared following the general procedure described in Example 1 and illustrated in FIG. 1. A larger (250 g) batch was prepared in the same manner. The specific processing parameters and microsphere physical characteristics are detailed in Table 3.

TABLE 3

| | |
|---|---|
| Batch Purpose | Month Two of Blend |

| | | |
|---|---|---|
| Batch No. | Lot 93 | Lot 158 |
| Batch Size | 20 g batch | 250 g batch |
| Symbol | Δ | ⋄ |

| | |
|---|---|
| Co-Monomer | 85:15 |
| Ratio | |
| Polymer IV | 0.27 |
| (dL/g) | |
| Polymer | Acid |
| Endcap | |
| Mixing Speed | 2250 |
| (RPM) | |
| Target Drug | 14 |
| Load (%) | |

| Drug Load | | | 10.8 | 11.4 |
|---|---|---|---|---|
| (%) | | | | |
| Encapsulation | | | 77 | 81 |
| Efficiency (%) | | | | |
| | Particle | Dᵥ10 | 43 | 25 |
| | Size | Dᵥ50 | 74 | 59 |
| | (μm) | Dᵥ90 | 117 | 100 |

| | | |
|---|---|---|
| Sample MW | 27.1 | 27.9 |
| (kDa) | | |
| Polymer MW | 27.9 | 28.4 |
| (kDa) | | |

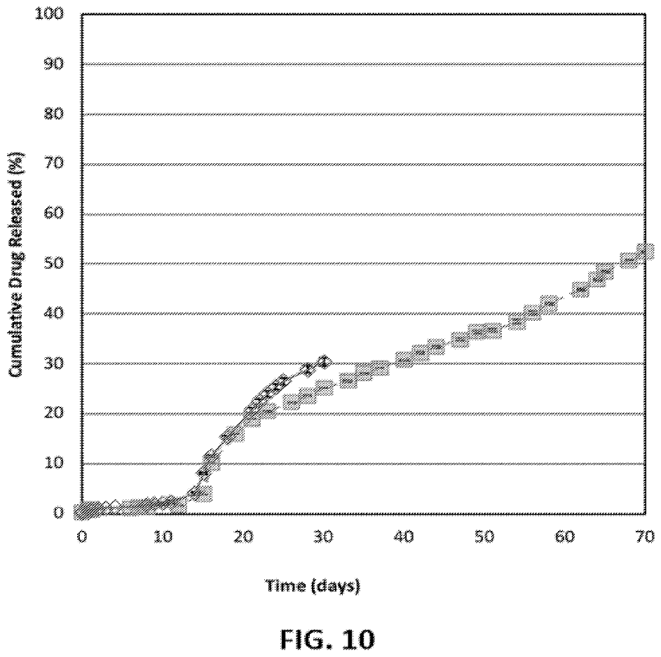

FIG. 10 is a graph showing an amount of octreotide acetate released in vitro over time from the smaller and larger scale batches of month two octreotide acetate-encapsulated polymer microspheres.

In use, the microspheres may be suspended in a diluent for administration (injection). The diluent may generally contain a thickening agent, a tonicity agent, and a wetting agent. The thickening agent may include carboxymethyl cellulose-sodium (CMC-Na) or other suitable compounds. In one aspect, the polymer microspheres do not comprise a cellulose-derived material. An appropriate viscosity grade and suitable concentration of CMC-Na may be selected so that the viscosity of the diluent is 3 cps or higher. Generally, a viscosity of about 10 cps is suitable; however, a higher viscosity diluent may be preferred for larger microspheres to minimize the settling of microspheres in the suspension.

Uniform microsphere suspension without particle settling will result in a consistent delivered dose during drug administration by injection. To have a tonicity of the diluent closer to the biological system, about 290 milliosmole (mOsm), solutes such as mannitol, sodium chloride, or any other acceptable salt may be used. The diluent may also contain a buffer salt to maintain the pH of the composition. Typically, the pH is maintained around a physiologically relevant pH by adjusting the buffer content as needed (pH about 7 to about 8).

The aspects disclosed herein are not intended to be exhaustive or to be limiting. A skilled artisan would acknowledge that other aspects or modifications to instant aspects can be made without departing from the spirit or scope of the invention. The aspects of the present disclosure, as generally described herein and illustrated in the figures, can be arranged, substituted, combined, separated, and designed in a wide variety of different configurations, all of which are contemplated herein.

Unless otherwise specified, “a,” “an,” “the,” “one or more of,” and “at least one” are used interchangeably. The singular forms “a”, “an,” and “the” are inclusive of their plural forms. The recitations of numerical ranges by endpoints include all numbers subsumed within that range (e.g., 1 to 5 includes 1, 1.5, 2, 2.75, 3, 3.80, 4, 5, etc.). The terms “comprising” and “including” are intended to be equivalent and open-ended. The phrase “consisting essentially of” means that the composition or method may include additional ingredients and/or steps, but only if the additional ingredients and/or steps do not materially alter the basic and novel characteristics of the claimed composition or method. The phrase “selected from the group consisting of” is meant to include mixtures of the listed group.

When reference is made to the term “each,” it is not meant to mean “each and every, without exception.” For example, if reference is made to microsphere formulation comprising polymer microspheres, and “each polymer microsphere” is said to have a particular octreotide acetate content, if there are 10 polymer microspheres, and two or more of the polymer microspheres have the particular octreotide acetate content, then that subset of two or more polymer microspheres is intended to meet the limitation.

The term “about” in conjunction with a number is simply shorthand and is intended to include ±10% of the number. This is true whether “about” is modifying a stand-alone number or modifying a number at either or both ends of a range of numbers. In other words, “about 10” means from 9 to 11. Likewise, “about 10 to about 20” contemplates 9 to 22 and 11 to 18. In the absence of the term “about,” the exact number is intended. In other words, “10” means 10.

1. A mixed release profile polymer microsphere formulation, comprising:
(A) first polymer microspheres, each first polymer microsphere comprising:
(i) octreotide or a pharmaceutically acceptable salt thereof; and
(ii) a first biodegradable polymer comprising a poly(D,L-lactide-co-glycolide) having a lactide: glycolide ratio of about 75:25,
wherein each first polymer microsphere comprises a drug load of octreotide of between about 7% to 10% by weight of the first polymer microsphere,
wherein the first polymer microspheres have a particle size of about 50 μm to about 90 μm (D₅₀), and
wherein the first polymer microspheres are characterized by a release of octreotide above a therapeutic level between about 5 days and about 40 days; and
(B) second polymer microspheres, each second polymer microsphere comprising:
(i) octreotide; and
(ii) a second biodegradable polymer comprising a poly(D,L-lactide-co-glycolide) having a lactide: glycolide ratio of about 85:15,
wherein each second polymer microsphere comprises a drug load of octreotide of between about 10% to 14% by weight of the first polymer microsphere,
wherein the second polymer microspheres have a particle size of about 50 μm to about 90 μm (D₅₀), and
wherein the second polymer microspheres are characterized by a release of octreotide above a therapeutic level between about 20 days and about 60 days.

2. The mixed release profile polymer microsphere formulation of claim 1, wherein the octreotide comprises octreotide acetate.

3. The mixed release profile polymer microsphere formulation of claim 1, wherein the first and second biodegradable polymers are acid-terminated.

4. The mixed release profile polymer microsphere formulation of claim 1, wherein the first biodegradable polymer has an inherent viscosity between about 0.1 dL/g and 0.3 dL/g.

5. (canceled)

6. The mixed release profile polymer microsphere formulation of claim 1, wherein the second biodegradable polymer has an inherent viscosity between about 0.2 dL/g and 0.4 dL/g.

7. (canceled)

8. The mixed release profile polymer microsphere formulation of claim 1, further characterized in that at least 75% the total octreotide is released over a period of about 60 days of injection into a subject, but not more than about 20% of the total octreotide has been released within about 24 hours of injection into the subject.

9. The mixed release profile polymer microsphere formulation of claim 1, further characterized in that the first polymer microspheres exhibit a peak release of octreotide between about 15 days and about 25 days, and the second polymer microspheres exhibit a peak release of octreotide between about 35 days and about 50 days.

10. A pharmaceutical composition comprising the mixed release profile polymer microsphere formulation of claim 1.

11. A method for making the mixed release profile microsphere formulation of claim 1, the method comprising:
(A) mixing:
(i) a first biodegradable polymer comprising a poly(D,L-lactide-co-glycolide) having a co-monomer ratio of about 75:25 in a first polymer solvent system to form a first polymer solution;
(ii) octreotide or a pharmaceutically salt thereof in a first drug solvent system to form a first octreotide solution; and
(iii) the first polymer solution with the first octreotide solution to form a first dispersed phase;
(B) mixing:
(i) a second biodegradable polymer comprising a poly(D,L-lactide-co-glycolide) having a co-monomer ratio of about 85:15 in a second polymer solvent system to form a second polymer solution;
(ii) octreotide or a pharmaceutically salt thereof in a second drug solvent system to form a second octreotide solution; and
(iii) the second polymer solution with the second octreotide solution to form a second dispersed phase;
(C) mixing:
(i) water; and
(ii) a surfactant, to form a continuous phase;
(D) combining the first dispersed phase with a first portion of the continuous phase in a first homogenizer to form a first polymer microsphere formulation having a first release profile;
(E) combining the second dispersed phase with a second portion of the continuous phase in a second homogenizer to form a second polymer microsphere formulation having a second release profile; and
(F) combining the first polymer microsphere formulation with the second polymer microsphere formulation to form the mixed release profile microsphere formulation.

12. The method of claim 11, wherein the first polymer solvent system and the second polymer solvent system are each dichloromethane.

13. The method of claim 11, wherein the first drug solvent system and the second drug solvent system are each a mixture of methanol and acetic acid.

14. The method of claim 11, wherein:
(i) the first and second biodegradable polymers are acid-terminated;
(ii) the first biodegradable polymer has an inherent viscosity between about 0.1 dL/g and 0.3 dL/g; and
(iii) the second biodegradable polymer has an inherent viscosity between about 0.2 dL/g and 0.4 dL/g.

15. A method for treating acromegaly or diarrhea, the method comprising administering by intramuscular injection to a patient in need thereof a mixed release profile polymer microsphere formulation according to claim 1.

16. (canceled)

17. (canceled)

18. A kit, comprising a mixed release profile polymer microsphere formulation according to claim 1.
